(19) United States
(12) Patent Application Publication
(10) Pub. No.: US 2008/0199956 A1
(43) Pub. Date: Aug. 21, 2008

(54) Storage Medium For Cells

(75) Inventors: Lubomir Arseniev, Hannover (DE); Krassimira Alexandrova, Hannover (DE); Marc Barthold, Hannover (DE); Carsten Griesel, Hannover (DE); Hans-Gerd Heuft, Ronnenberg (DE); Sabine Kafert-Kasting, Hannover (DE); Christoph Priesner, Hannover (DE)

Correspondence Address: DARBY & DARBY P.C., P.O. BOX 770, Church Street Station, New York, NY, 10008-0770, US

(73) Assignee: Cytonet GmbH & Co. KG, Weinheim/Bergstrasse (DE)

(21) Appl. No.: 11/994,231

(22) PCT Filed: Jun. 30, 2006
(86) PCT No.: PCT/EP2006/006401

§ 371 Date: Mar. 26, 2008

(30) Foreign Application Priority Data
Jul. 1, 2005 (DE) 10 2005 031 532.1

(51) Int. Cl. C12N 5/06

(52) U.S. Cl. 435/374; 435/407

(57) ABSTRACT

The invention relates to a method for storages of human cells in particular, cell media for storage of cells and the use of such cell media for storage of cells, wherein the vitality of the cells is preserved during the storage.

This application is a U.S. national phase application under 35 U.S.C. §371 of International Patent Application No. PCT/EP2006/006401 filed Jun. 30, 2006, which claims the benefit of priority to German Patent Application No. DE 10 2005 031 532.1 filed Jul. 1, 2005, the disclosures of all of which are hereby incorporated by reference in their entireties. The International Application was published in German on Jan. 11, 2007 as WO 2007/003382.

### FIELD OF THE INVENTION

The present invention relates to methods for storing human cells in particular, cell media for storage of cells and use of such cell media for storage of cells, such that the vitality of the cells is preserved during storage.

### BACKGROUND

Cryopreservation is a method often used for storage of viable cells for long periods of time. The cryopreservation techniques have been constantly further developed and improved upon in the course of the last decade. There is a demand for storing cells even of a short period of time, e.g., directly after isolation from an organ, before freezing, or during or after thawing the cells after cryopreservation. It is often necessary for the cells to be administered back to the subject again, in particular a human being, after such storage. This administration of cells may be accomplished by intravenous administration of the cells, for example. Administration of cells stored by cryopreservation in particular to a human may be used especially for a clinical application, in particular as a therapeutic application. For example human liver cell transplants have been administered intravenously for therapeutic use. However, the freezing process and thawing process and the in vitro storage either prior to that or immediately following remain a problem with regard to the vitality and viable cell count of the cells to be stored, especially in clinical use.

The present invention is based on the technical problem of providing a cell medium that is simple to prepare and is as inexpensive as possible for storage of animal cells, preferably human cells, especially liver cells or liver cell transplants. These cells should preferably be stored in conjunction with cryopreservation before freezing and/or after thawing. Therefore the object of the present invention is also to provide a cell medium that allows short-term storage of animal cells, preferably human cells, especially preferably liver cells or liver cell transplants with the least possible loss of vitality and viable cell count of the cells and thus constitutes an alternative to the known cell media.

In conjunction with cryopreservation, cell media for storage and rethawing are known. However, they contain ingredients such as 5% to 20% fetal calf serum (FCS), which are not allowed by law or are unsuitable for administration to humans, in particular by intravenous injection. The present invention is therefore additionally based on the technical problem of making available a medium that allows risk-free administration of the cells to an animal, in particular a human, with a minor loss of vitality and viable cell count of cells, preferably human cells, in particular liver cells or liver cell transplants.

For example, Custodiol® can be used successfully as a cell medium, e.g., for storage of thawed originally cryopreserved cells such as liver cells. It was developed as a preservative solution for organ transplantation of whole organs. However, Custodiol® is not allowed by law for intravenous injection into humans. Therefore the object of the present invention is also the technical problem of providing a cell medium for short-term shortage of cells, in particular in conjunction with cryopreservation, that is allowed for intravenous injection to humans and therefore the cells can be administered intravenously to a human in this cell medium after being thawed without requiring complex washing steps which would reduce the viable cell count.

The present invention solves the technical problem on which it is based essentially by providing a method for short-term storage of animal or human cells according to the patent claims. In a preferred method, the cells are stored in a liquid cell medium containing a salt solution selected in particular from a salt solution containing sodium chloride, sodium gluconate, sodium acetate, potassium chloride, magnesium chloride and sodium citrate and/or a salt solution containing Ringer's lactate and/or phosphate-buffered saline and/or an aqueous solution of a high-molecular sugar, in particular hydroxyethyl starch and glucoses and also serum albumin and/or blood plasma and storing the cells in this cell medium.

In preferred embodiments, the animal cells are mammalian cells. For example they may be porcine, bovine or murine cells. The cells are especially preferably human cells. According to the present invention, the term “cells” is understood to also include cell suspensions and cell transplants. The cells may all be of one type, but a composition and/or co-culture of different cell types may also be used.

The animal cells used according to the present invention are preferably cells for therapeutic administration. In another preferred embodiment, the animal cells are cells for in vitro culturing. The animals may be, for example, hepatocytes, islets of Langerhans (pancreas islets), chondrocytes, cartilage cells, nerve cells, keratinocytes or lymphocytes. The cells provided for storage in a cell medium are most preferably liver cells, in particular hepatocytes.

In conjunction with the present invention, the term liver cells is understood to refer to a mixture of cells consisting primarily of hepatocytes. However, they may also contain various amounts of lymphocytes. Endothelial cells and other nonparenchymatous cell groups may also be present.

The present invention does not include thawing and/or storage of thrombocytes, also known as blood platelets. Platelets are not animal cells in the sense of this invention.

In conjunction with this invention, short-term storage is understood to refer to in vitro storage in an inventive medium for a period of up to 24 hours, especially up to 10 hours, preferably up to 5 hours, especially up to 3 hours.

Cryconservation usually refers to long-term storage of cells at temperatures below 0° C., especially −20° C. to −200° C. The freezing rate is usually 1° C./min.

In conjunction with the present invention, a cell medium, also known as medium, thawing medium or culture medium, is understood to refer in particular to a liquid aqueous solution. The cell medium provides in vitro storage of viable cells while retaining the highest possible viable cell count, i.e., the lowest possible mortality rate of the cells and the highest possible vitality, also known to those skilled in the art as viability.

It is also preferred according to this invention for the ingredients of a cell medium used according to this invention to be allowed by law for administration, in particular intravenous injection, into an animal body, preferably a human body. According to this invention, after being stored in an inventive medium, the cells may be used for a clinical application, in particular a therapeutic application, or for in vitro experiments.

In conjunction with this invention, the term salt solution is understood to refer to an aqueous solution of one or more salts. A salt solution that is preferred according to the present invention contains sodium chloride, sodium gluconate, sodium acetate, potassium chloride, magnesium chloride and sodium citrate, such that the salts are dissolved in water. The water used is sterile water, preferably deionized water for injection.

Composol® and/or a solution corresponding to the composition of Composol® is a preferred salt solution according to this invention. Composol® is an aqueous solution of sodium chloride, sodium gluconate, sodium acetate, potassium chloride, magnesium chloride hexahydrate and sodium citrate. It contains 173 mmol/L sodium, 5 mmol/L potassium, 1.5 mmol/L magnesium, 98 mmol/L chloride, 10.9 mmol/L citrate, 27 mmol/L acetate and 23 mmol/L gluconate, preferably consisting thereof.

The salt solution according to this invention therefore preferably contains from 0.45 to 0.60 wt %, especially preferably 0.53 wt % sodium chloride, 0.45 to 0.55 wt %, especially preferably 0.50 wt % sodium gluconate, 0.20 to 0.25 wt %, especially preferably 0.22 wt % sodium acetate, 0.03 to 0.04 wt %, especially preferably 0.037 wt % potassium chloride, 0.025 to 0.035 wt %, especially preferably 0.031 wt % magnesium chloride hexahydrate and 0.31 to 0.33 wt %, especially preferably 0.32 wt % sodium citrate, preferably consisting thereof. The salt solution preferably has a pH of 7.0 to 7.4, especially preferably 7.2.

In another embodiment the salt solution is Ringer's lactate. Those skilled in the art are familiar with the usual composition of Ringer's lactate. Ringer's lactate is an aqueous solution containing sodium chloride, potassium chloride, calcium chloride and sodium lactate. Ringer's lactate used according to this invention has a pH of 7.0 to 7.4, preferably 7.2.

In another embodiment, the salt solution is phosphate-buffered saline (PBS). Those skilled in the art are familiar with the usual composition of PBS. PBS used according to the present invention has a pH of 7.0 to 7.4, preferably 7.2.

In another embodiment, an ingredient of the medium is an aqueous solution of at least one high-molecular sugar. Inventive high-molecular sugars include, for example, dextran, also dextran solutions such as perfadex, hydroxyethyl starch (HES) and derivatives of hydroxyethyl starch. Hydroxyethyl starch is the preferred high-molecular sugar according to this invention.

According to this invention glucose is added to the cell medium in solid form or as an aqueous solution.

According to this invention, the serum albumin is added to the cell medium in dissolved or liquid form. Serum albumin may be, for example, fetal calf serum (FCS), bovine serum albumin (BSA) or human serum albumin (HSA). Human serum albumin is preferred according to this invention because it is allowed for intravenous injection to humans.

In another embodiment, serum albumin is supplemented or replaced by blood plasma. According to this invention, human blood plasma, especially autologous, is preferred. Plasma and/or HSA is especially used as a cryoprotector, preferably alone or in combination with other known cryoprotectors, especially DMSO.

In another preferred embodiment, the medium use according to this invention contains at least one other ingredient selected from amino acids, hormones, vitamins, provitamins and antiapoptotically active substances.

Those skilled in the art will select the ingredients of the medium corriposition depending on the area for use in accordance with their technical knowledge after being instructed by the inventive teaching to use the inventive ingredients and the inventive concentrations.

The inventive method is preferably performed using a cell medium like that defined above with a pH of 6.4 to 8, preferably 7.0 to 7.4, especially 7.2.

After thawing from cryopreservation in the inventive cell medium, the cells are preferably washed, separated from the preservation medium and the inventive cell medium and then placed in fresh inventive cell medium for storage. The cells are then preferably separated by centrifugation.

This method is preferably carried out at a temperature of 2 to 40° C. The cell medium preferably has a temperature of 2 to 40° C. The method is especially preferably carried out at 4° C. and/or the medium used has a temperature of 4° C. In another preferred variant, the method is carried out at 10° C. and/or the medium used has a temperature of 10° C. In another variant that is especially preferred, the method is carried out at 25° C. and/or the medium used has a temperature of 25° C. In another variant that is especially preferred, the method is carried out at 37° C. and/or the medium used has a temperature of 37° C.

The subject matter of the present invention is of course also the special cell medium for short-term storage of animal or human cells, in particular cells that have been thawed after cryopreservation, especially a liquid cell medium that contains sodium chloride, sodium gluconate, sodium acetate, potassium chloride, magnesium chloride and sodium citrate as well as glucose and also serum albumin and/or blood plasma and in which the aforementioned ingredients are dissolved in water.

A preferred cell medium consists of sodium chloride, sodium gluconate, sodium acetate, potassium chloride, magnesium chloride hexahydrate and sodium citrate as well as glucose and also serum albumin and/or blood plasma, such that the aforementioned ingredients are dissolved in water. An especially preferred cell medium contains 0.45 to 0.60 wt % sodium chloride, 0.45 to 0.55 wt % sodium gluconate, 0.20 to 0.25 wt % sodium acetate, 0.03 to 0.04 wt % potassium chloride, 0.025 to 0.035 wt % magnesium chloride hexahydrate and 0.31 to 0.33 wt % sodium citrate plus glucose and also serum albumin and/or blood plasma, the latter constituents being dissolved in water.

The cell medium preferably contains 0.01 to 0.5 wt % glucose, especially preferably 0.1 to 0.2 wt % glucose.

It is preferred according to this invention for the cell medium to contain human serum albumin. The human serum albumin may be replaced according to this invention by human blood plasma, especially autologous blood plasma. In a preferred embodiment, the medium contains 0.5 to 0.50 wt % serum albumin, especially preferably 3 to 5 wt % serum albumin.

In another preferred embodiment, the medium contains 0.5 to 50 wt % blood plasma, especially preferably from 3 to 5 wt % blood plasma.

The inventive cell medium preferably has a pH of 6.0 to 8, preferably 7.0 to 7.4, especially 7.2.

The components of an inventive cell medium are preferably allowed by law for application, in particular intravenous administration to an animal body, preferably a human body.

Combinations of inventive embodiments of the inventive method and/or the inventive cell medium are also preferred according to this invention.

The invention also comprises the use of the aforementioned inventive cell medium for short-term storage of animal cells thawed after cryopreservation, especially by the inventive method.

### BRIEF DESCRIPTION OF THE DRAWINGS

FIG. 1: FIGS. 1(a)-1(f) show the vitality of the stored cells (after cryopreservation) as a function of the storage time, the storage medium used and the storage temperature. (a) HES vitality; (b) Custodiol vitality; (c) Ringer's lactate vitality; (d) PBS vitality; (e) Composol vitality; and (f) Human plasma vitality. Legend: HH142: number of the individual cell preparation (batch), warm: storage temperature of 18° C., cold: storage temperature of 4° C. HES: medium 2. PBS: medium 5. Custodiol®: medium 1. Composol®: medium 4. Ringer's lactate: medium 3. Human plasma: medium 6.

FIG. 2: FIGS. 2(a)-2(f) show the recovery of stored cells (before cryopreservation) as a function of storage time, the storage medium used and the storage temperature. (a) HES—recovery before freezing; (b) Custodiol—recovery before freezing; (c) Ringer's lactate—recovery before freezing; (d) PBS—recovery before freezing; (e) Composol—recovery before freezing; and (f) Human plasma—recovery before freezing. See FIG. 1 for Legend.

FIG. 3: FIGS. 3(a)-3(d) show the vitality of stored cells (after cryopreservation) as a function of storage time, the storage medium used and the storage temperature. (a) HH142 vitality; (b) HH051 vitality; (c) HH122 vitality; and (d) HH123 vitality. See FIG. 1 for Legend.

FIG. 4: FIGS. 4(a)-4(d) show the viable cell count (VCC) of stored cells (after cryopreservation) as a function of storage time and the storage medium used. (a) HH142—VCC; (b) HH051—VCC; (c) HH122—VCC; and (d) HH123—VCC. See FIG. 1 for Legend.

FIG. 5: FIGS. 5(a) and 5(b) show the viable cell count (VCC) of stored cells (before cryopreservation) as a function of storage time and the storage medium used; median over three individual cell preparations (batches). See FIG. 1 for Legend.

FIG. 6: FIGS. 6(a) and 6(b) show the viable cell count (VCC) of stored cells (before cryopreservation) as a function of storage time and the storage medium used; median over four individual cell preparations (batches). See FIG. 1 for Legend.

FIG. 7: FIGS. 7(a) and 7(b) show the recovery of viable cell count (VCC) of stored cells (before cryopreservation) as a function of storage time and the storage medium used; median over three individual cell preparations (batches). FIG. 7(c) shows the comparison with Custodiol (=100%), average over four storage times; median over three individual cell preparations (batches). See FIG. 1 for Legend.

### EXAMPLE

Cryopreserved liver cells from special cryobags/cryotubes were used. Since the number of cryotubes available from an individual cell preparation was not sufficient for the extent of the study, they were used only for comparison of results. All the preparation steps were performed under aseptic conditions. The cell suspension was completely thawed before adding the cell medium. The cryotubes were thawed at a point independent in time from the thawing of the bags. The cell medium was slowly added to the cells which had already been set out.

To achieve comparable results, the various experiments were conducted in parallel by at least two people, optionally three people.

The following thawing media were prepared:
Medium 1: Custodiolan (comparative example)
Medium 2: Hydroxyethyl starch (HES)+1 g/L glucose+4% HSA (according to the present invention)
Medium 3: Ringer's lactate+1 g/L glucose+4% HSA (according to the present invention)
Medium 4: Composol®+1 g/L glucose+4% HSA (according to the present invention)
Medium 5: Phosphate-buffered saline (PBS)+1 g/L glucose+4% HAS (according to the present invention)
Medium 6: Human blood plasma (comparative example)

Approximately 50 mL cell medium was needed per run (one thawed bag). Human blood plasma was thawed in a warm water bath at 37° C.

In three experiments, the media were warmed for at least 15 minutes in a warm water bath at 37° C. (=“warm”). In two experiments the cell media were cooled under refrigeration for at least 15 minutes (=“cold”).

Six 50 mL tubes labeled according to the solutions were set out and stored at room temperature.

In the warm experiments, the cells were centrifuged at 50 g's for 5 minutes at 18° C. and in the cold experiments they were centrifuged at 50 g's for 5 minutes at 4° C.

Cryobags were cautiously removed from the aluminum cassette. The cryosuspension was thawed for 2 to 5 minutes while shaking constantly until no more ice crystals were visible. The entire process took no longer than 5 minutes. The yellow cap was removed from the bag and the septum was punctured with the puncture needle of a Benjamix. A 60 mL syringe was connected to the Benjamix of the cryobag and 60 mL cell suspension was withdrawn.

10 mL portions of cell suspension were placed in each of the six prepared tubes. To this was slowly added 40 mL of the corresponding cell media. The entire process did not take more than 5 minutes.

After centrifugation, the cells were washed. The supernatants were removed by using a Pasteur pipette. The cell pellets were completely resuspended in 5 mL of the corresponding cell medium by shaking cautiously.

The cells were counted according to cell count and vitality and a liver cell culture was prepared after each cell count.

For a total storage time of 3 hours, the last two steps were repeated every 60 minutes.

Only liver cells with a vitality greater than 40% before cryopreservation were used. The experiment was repeated at least three times with different individual cell preparations (batches). At least one bag was thawed per batch. The results of thawing cryotubes of the respective batches were used for comparison.

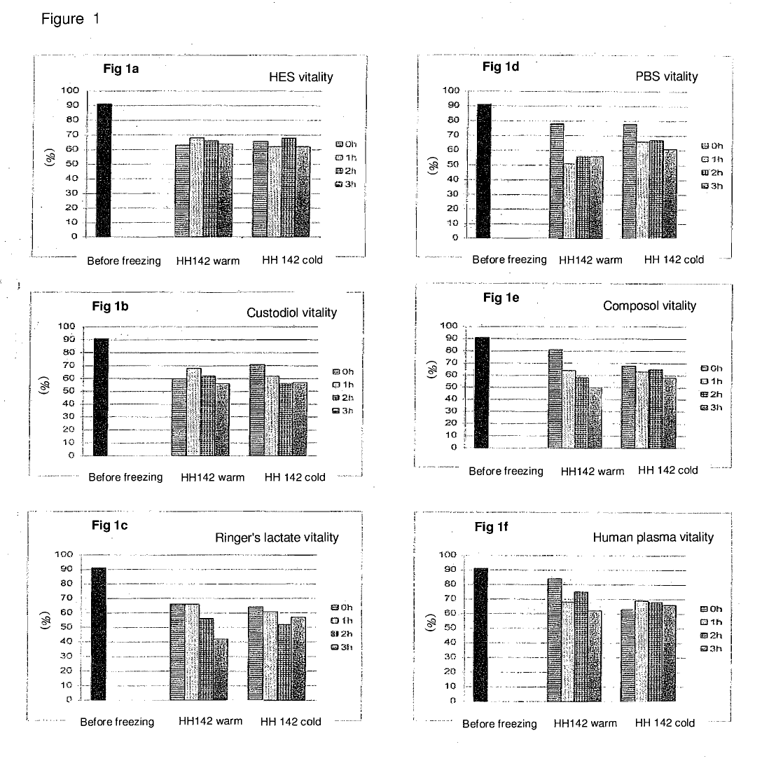

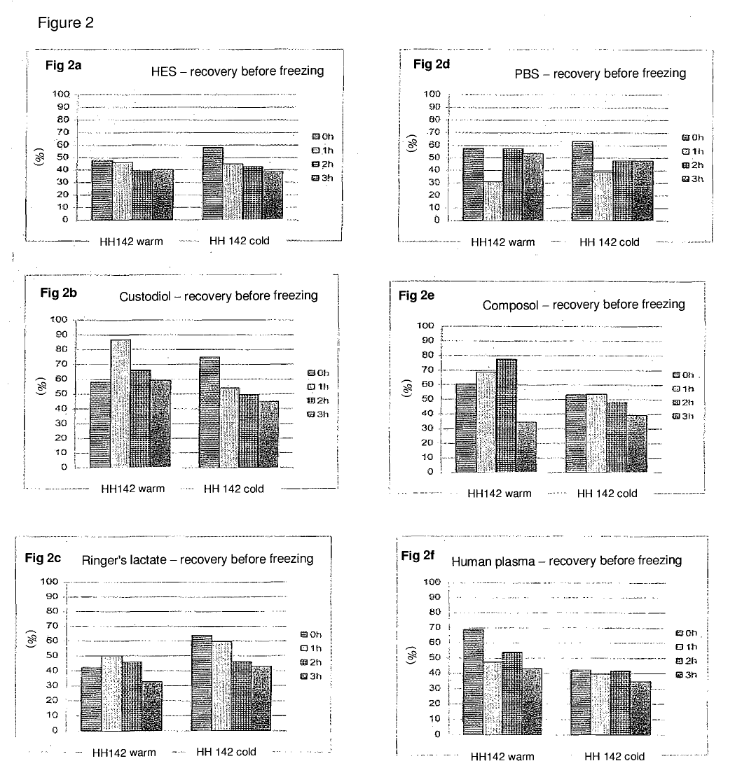

For individual cell preparations (batches) were tested with regard to the effect of the thawing solution. These results were tested with regard to the parameters of vitality and viable cell count (VCC) after thawing and vitality and viable cell count preserved during storage in suspension. The temperature of the medium and for centrifugation was set at 4° C. for the liver cell transplants. The experiment with batch HH142 was performed once “warm” and then was repeated “cold” (see FIG. 1 and FIG. 2). For further analysis, the averages of the two experiments were used and referred to HH142.

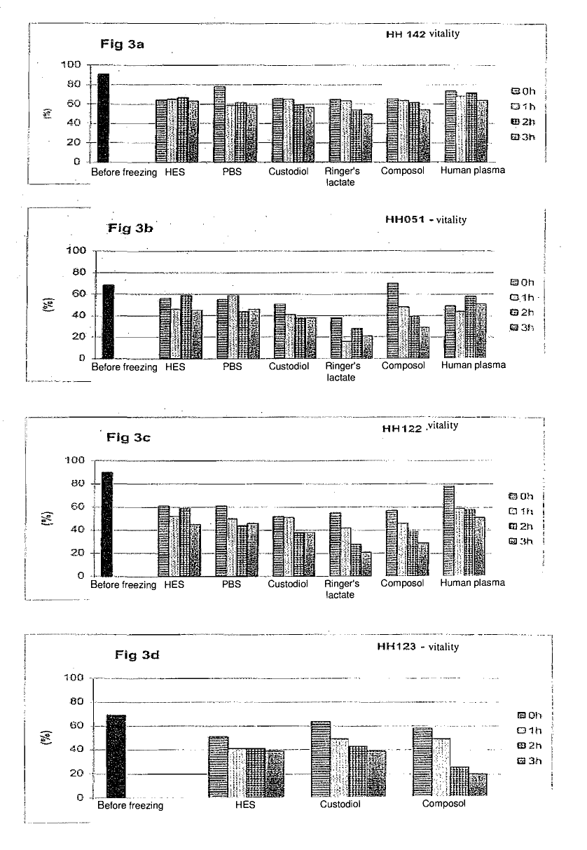

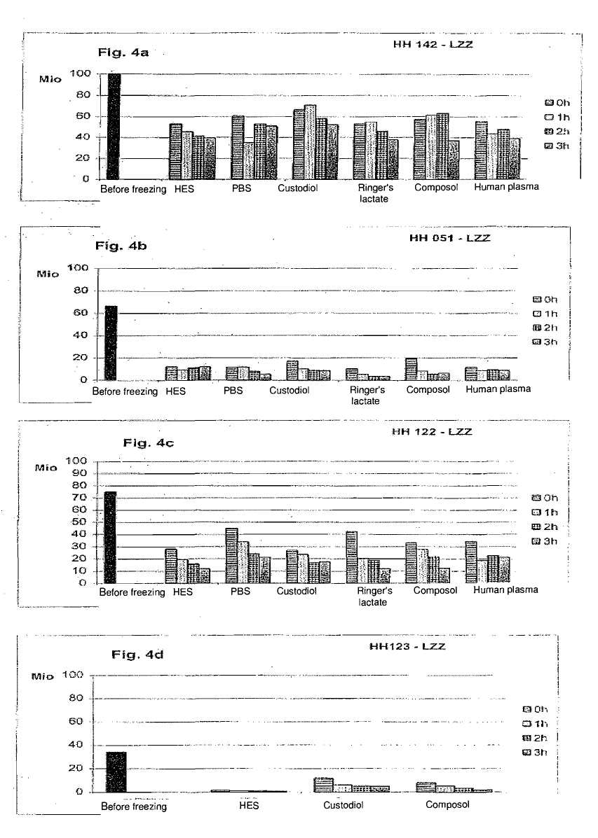

The individual results are shown in FIGS. 3 and 4.

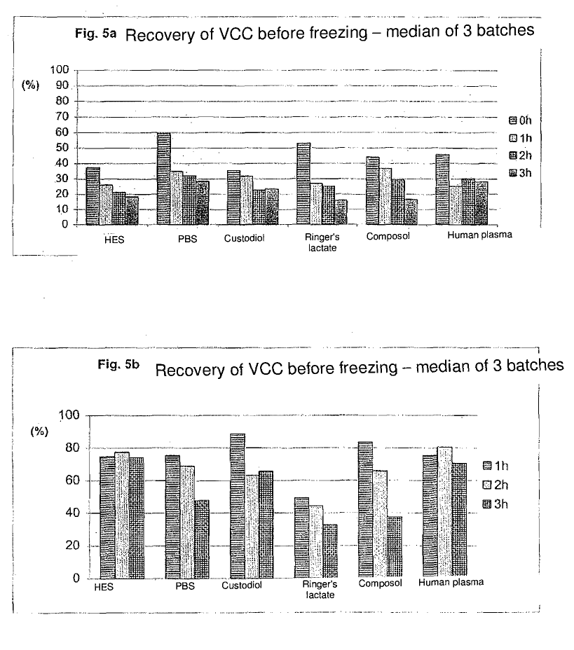

To ensure a batch-independent analysis, the recovery and viable cell count parameters for storage in the tested solutions were defined. This was based on two different starting points: first, the recovery based on the viable cell count before freezing (FIG. 5a), secondly, the recovery based on the viable cell count after washing (hour 0 in the specific storage medium) (FIG. 5b). For comparison of the five solutions, median values for the aforementioned parameters were calculated based on the three batches tested and plotted in the graph in FIG. 5.

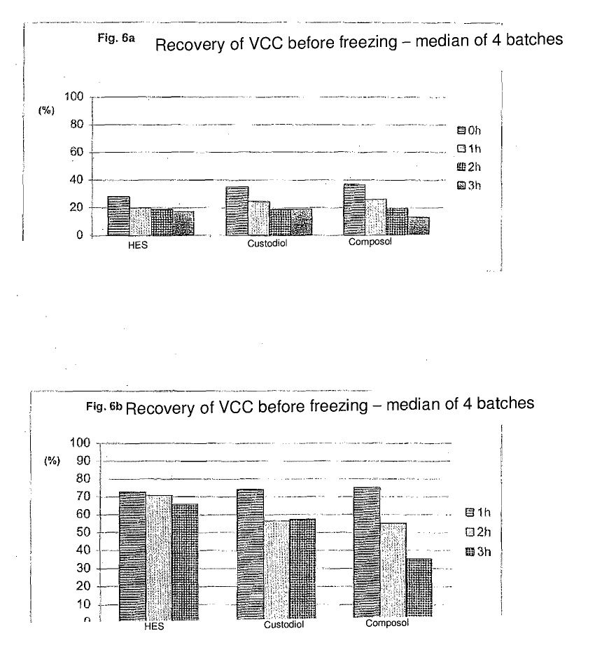

The two selected solutions—Composol® and HES—were analyzed additionally on the basis of the median values of four batches, with the results depicted graphically in FIG. 6.

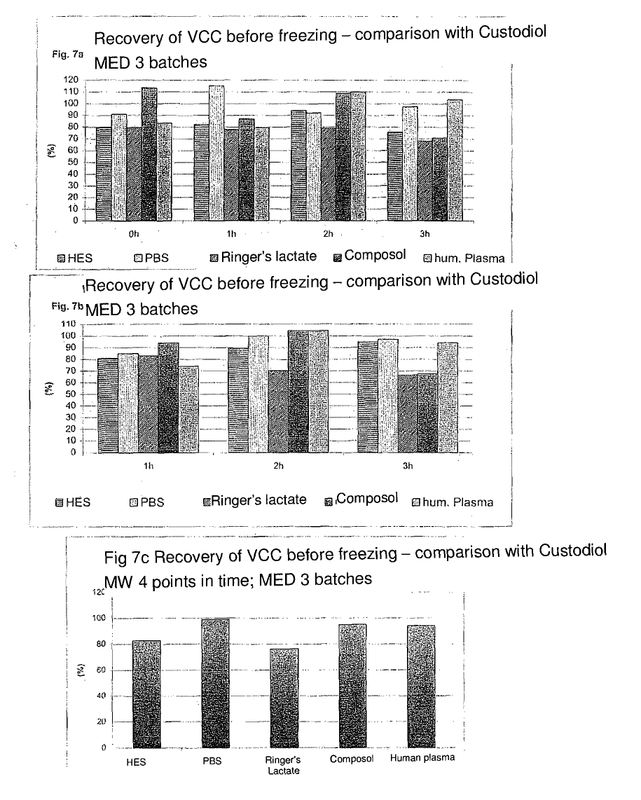

For a better analysis of the results shown here, the following approach was used. The parameter of recovery of frozen VCC was tested on Custodiol® 100%. For the five batches tested (comparative example/inventive examples) the percentage deviation in this parameter in comparison with Custodiol® was calculated (see FIG. 7a and FIG. 7b).

Immediately after washing the cells, three of the solutions (PBS, Composol® and HES) showed a comparable agreement (greater than 80%) with Custodiol® with regard to the recovery of VCC as a parameters. Composol® yielded even better results than Custodiol® (recovery greater than 100%). After one hour, PBS had the best results, but after two hours, Composol® and human plasma again had approximately 10% better median results than Custodiol®.

For further comparison, another analysis was performed. For each solution the average of all four points in time was calculated (hours 0 through 3, FIG. 7c).

Regardless of the time elapsed in the experiment, HES and Ringer's lactate showed approximately 20% loss of VCC in comparison with Custodiol®, but PBS, Composol® and human plasma showed results comparable to those obtained with Custodiol® (deviation less than 10%).

All references cited and/or discussed in this specification are incorporated herein by reference in their entireties and to the same extent as if each reference was individually incorporated by reference.

1. A method for short-term storage of animal cells, comprising placing the cells in a liquid cell medium comprising:
a) a salt solution selected from:
a solution comprising sodium chloride, sodium gluconate, sodium acetate, potassium chloride, magnesium chlorides and sodium citrate,
Ringer's lactates and
phosphate-buffered saline (PBS);
and/or an aqueous solution of a high-molecular sugar;
b) glucose; and
c) serum albumin and/or blood plasma; and
storing the cells in the liquid cell medium.

2. The method according to claim 1, wherein the cells are cryopreserved cells and are thawed and then after thawing are washed in a cell medium, separated from the cell medium, and then placed in a fresh cell medium for storage.

3. The method according to claim 1, wherein the cell medium has a temperature of 2 to 40° C.

4. The method according to claim 1, wherein the cells are liver cells.

5. The method according to claim 4, wherein the cells are hepatocytes.

6. The method according to claim 1, wherein the cells are mammalian cells.

7. The method according to claim 1, wherein the cells are human cells.

8. The method according to claim 1, wherein the cells are cells for therapeutic administration.

9. The method according to claim 1, wherein the cells are for in vitro culturing.

10. (canceled)

11. A cell medium for short-term storage of animal cells, wherein the cell medium comprises the following ingredients:
a) sodium chloride, sodium gluconate, sodium acetate, potassium chloride, magnesium chlorides and sodium citrate;
b) glucose; and
c) serum albumin and/or blood plasma;
wherein the ingredients are dissolved in water.

12. The cell medium according to claim 11, wherein the serum albumin is human serum albumin.

13. The cell medium according to claim 11, wherein the blood plasma is human blood plasma.

14. The cell medium according to claim 11, wherein the cell medium comprises:
0.45 to 0.60 wt % sodium chloride;
0.45 to 0.55 wt % sodium gluconate;
0.20 to 0.25 wt % sodium acetate;
0.03 to 0.04 wt % potassium chloride;
0.025 to 0.035 wt % magnesium chloride hexahydrate; and
0.31 to 0.33 wt % sodium citrate.

15. The cell medium according to claim 11, wherein the cell medium contains 0.01 to 0.5 wt % glucose.

16. The cell medium according to claim 11, wherein the cell medium contains 0.5 to 50 wt % serum albumin.

17. The cell medium according to claim 11, wherein the cell medium contains 0.5 to 50 wt % blood plasma.

18. The cell medium according to claim 11, wherein the cell medium has a pH of 6.4 to 8.

19. The cell medium according to claim 11 comprising at least one additional ingredient selected from amino acids, hormones, vitamins, provitamins, and antiapoptotic substance.

20. The method of claim 1, wherein the salt solution contains sodium chloride, sodium gluconate, sodium acetate, potassium chloride, magnesium chloride and sodium citrate.

21. The method of claim 1, wherein the high-molecular sugar is hydroxyethyl starch.

22. The method of claim 15, wherein the cell medium contains 0.1 to 0.2 wt % glucose.

23. The cell medium of claim 16, wherein the cell medium contains 3 to 5 wt % serum albumin.

24. The cell medium of claim 17, wherein the cell medium contains 3 to 5 wt % blood plasma.

25. The cell medium of claim 18, wherein the cell medium has a pH of 7.0 to 7.4.
